(19) United States
(12) Patent Application Publication
(10) Pub. No.: US 2007/0258916 A1
(43) Pub. Date: Nov. 8, 2007

(54) Oral compositions for treating tooth hypersensitivity

(75) Inventors: Jack L. Ferracane, Beaverton, OR (US); John C. Mitchell, Beaverton, OR (US)

Correspondence Address: KLARQUIST SPARKMAN, LLP, 121 SW SALMON STREET, SUITE 1600, PORTLAND, OR, 97204, US

(73) Assignee: Oregon Health & Science University

(21) Appl. No.: 11/787,598

(22) Filed: Apr. 16, 2007

Related U.S. Application Data
(60) Provisional application No. 60/792,288, filed on Apr. 14, 2006.

(51) Int. Cl. A61K 8/24

(52) U.S. Cl. 424/57

(57) ABSTRACT

Oral Compositions for Treating Tooth Hypersensitivity Disclosed herein are oral compositions for decreasing tooth hypersensitivity. In one aspect, the compositions induce remineralization of dentine using bioactive glass, thereby reducing tooth sensitivity.

### CROSS REFERENCE TO RELATED APPLICATION

This claims the benefit of the earlier filing date of U.S. Provisional Patent Application No. 60/792,288, filed Apr. 14, 2006.

### FIELD

This disclosure concerns tooth desensitizers, and in particular concerns a biomimetic approach to desensitizing hypersensitive teeth.

### BACKGROUND

Tooth hypersensitivity is a common problem that chronically affects millions of adults in the United States. It is estimated that between about 8 and 35% of adults in the United States have at least one or more hypersensitive teeth that are painful in response to such stimuli as cold, heat, air or sugary foods.

Tooth hypersensitivity is believed to be related to the general increase in exposed root surfaces of teeth as a result of periodontal disease, toothbrush abrasion, or cyclic loading fatigue of the thin enamel near the dento-enamel junction. When root surfaces are exposed, dentinal tubules are also exposed. Dentinal tubules are naturally present in the dentinal layer of the tooth and they function to provide for an osmotic flow between the inner pulp region of the tooth and the outer root surfaces.

The hydrodynamic theory for tooth hypersensitivity maintains that open exposed dentinal tubules allow fluid flow through the tubules. This flow excites the nerve endings in the dental pulp. Occlusion of the dentinal tubules of a sensitive tooth by resin infiltration, varnish coat, or crystalline precipitation results in a reduction or elimination of the hypersensitivity. The duration of relief, however, is quite variable. Hypersensitivity usually reappears following toothbrush abrasion, presence of acid in the mouth, or degradation of the coating material.

Toothpastes containing potassium nitrate have been used to desensitize the nerve directly. Sensitivity toothpastes containing strontium chloride have also been used to help form crystals that cover the pores in exposed roots so that stimuli can not reach the exposed nerve. Desensitizing dentifrices with potassium oxalate have also been found to provide temporary tubule occlusion. Potassium oxalate is thought to react with the smear layer to increase its resistance to acid attack as well as reduce its permeability. It is thought that the crystals produced when dentin is treated with potassium oxalate are calcium oxalate.

However these prior approaches use biologically inactive inorganic or organic components that occlude the open tubules for a limited time period. Normal activities such as eating and toothbrushing remove the materials from the tubules and allow resumed fluid flow and tooth sensitivity.

### SUMMARY

The composition and methods disclosed herein deliver a densitizing agent to a tooth surface to relieve pain in people with sensitive teeth. The desensitizing agent includes a bioactive glass powder suspended in a liquid in which the glass is delivered to the tooth surface. The liquid may include organic acids or inorganic acids. The composition is suitable for being dispensed from a bottle and applied to the sensitive tooth surface with an applicator such as a brush or sponge. The material coats the tooth and obturates open dentinal tubules to provide immediate reduction in fluid movement through the exposed dentinal tubules that are eliciting the pain response. After more prolonged exposure, the bioactive glass component partially corrodes and precipitates a mineral that more completely and permanently occludes the tubules and the surface of the exposed dentin.

The disclosed compositions and methods provide an effective mechanism for the delivery of the bioactive glass to the surface of the tooth. Examples of the disclosed compositions are effective to deliver bioactive glass to the dentinal tubule and further to the dental pulp. This aids remineralization, in contrast to current treatments, which attempt to desenitize the tooth by exposing it to a nerve “deadening” agent (such as potassium nitrate) or by physically and inertly occluding the tubules (with potassium oxalate). Instead it is now possible to provide an inert occlusion of the open tubules which in time will undergo a chemical reaction when exposed to oral fluids that precipitates new mineral that more completely seals the open tubules and more uniformly covers the exposed dentin surface. Thus, in certain embodiments the bioactive glass is chemically incorporated into the tooth itself. Since the new material is formed directly upon and/or within pre-existing tooth mineral, and is of a similar composition to the tooth, the new mineral will firmly adhere to the tooth crystal structure and provide lasting relief and resistance to abrasion from food and toothbrushing.

The foregoing and other features and advantages of the invention will become more apparent from the following detailed description of a several embodiments which proceeds with reference to the accompanying figures.

### BRIEF DESCRIPTION OF THE DRAWINGS

FIG. 1 is a graph that demonstrates conductance (20 cm SBF) of BAG (15 wt % in glycerol) applied to dentin surface versus control after exposure to toothbrush abrasion, cola, grapefruit juice and coffee.

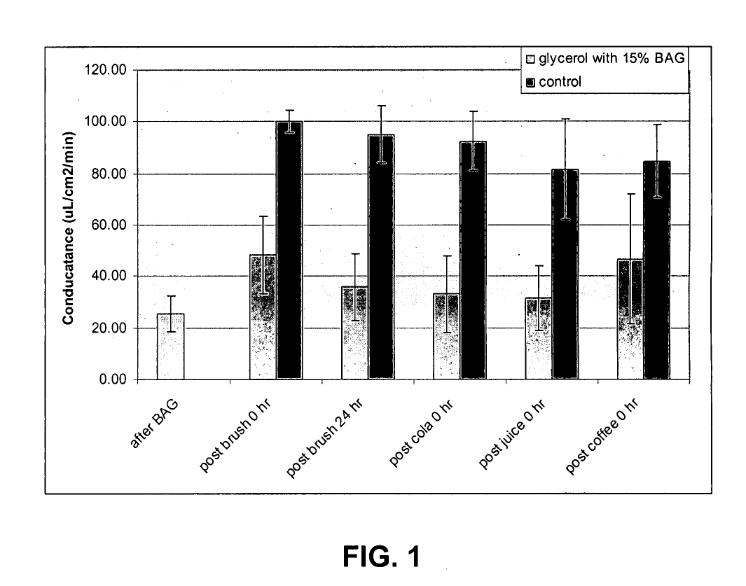

FIG. 2 is a graph that demonstrates tubule conductance following treatment of teeth with control, OX50 and BAG (bioactive glass).

FIG. 3 is a graph that demonstrates tubule conductance following treatment of teeth with BAG-65 mole % SiO₂/11 mole % CaO/4 mole % P₂O₅ in four different carriers (ethanol, HEMA, PPG and glycerol).

FIG. 4 is a series of photomicrographs that illustrate the surfaces of teeth after treatment of the teeth with BAG-65 mole % SiO₂/11 mole % CaO/4 mole % P₂O₅ using ethanol as a carrier.

FIG. 5 is a series of photomicrographs that illustrate the surfaces of teeth after treatment of the teeth with BAG-65 mole % SiO₂/11 mole % CaO/4 mole % P₂O₅ using glycerol as a carrier.

FIG. 6 is a series of photomicrographs that illustrate the surfaces of teeth after treatment of the teeth with BAG-65 mole % SiO₂/11 mole % CaO/4 mole % P₂O₅ using HEMA as a carrier.

FIG. 7 is a series of photomicrographs that illustrate the surfaces of teeth after treatment of the teeth with BAG-65 mole % SiO₂/11 mole % CaO/4 mole % P₂O₅ using propylene glycol as a carrier.

### DETAILED DESCRIPTION

“Bioactive glass” refers a group of surface reactive glass-ceramics that include the original bioactive glass, Bioglass®. The biocompatibility of these glasses has led them to be investigated extensively for use as implant materials in the human body to repair and replace diseased or damaged bone. There have been many variations on the original composition which was FDA approved and termed Bioglass®, which is also known as 45S5. As referred to herein, bioactive glasses are typically silicon dioxide-based compositions capable of forming hydroxycarbonate apatite when exposed to physiological fluids. Typically, the bioactive glasses for use in the presently disclosed compositions and methods have the following composition by molar percentage:

from about 50 to about 96 mole percent SiO₂;

from about 2 to about 50 mole percent CaO;

from about 2 to about 16 mole percent P₂O₅;

from about 0 to about 25 mole percent CaF₂; and

from about 0 to about 10% B₂O₃, or an equivalent thereof.

Examples of these and other compositions include:

45S5: 46.1 mol % SiO₂, 26.9 mol % CaO, 24.4 mol % Na₂O and 2.5 mol % P₂O₅.

58S: 60 mol % SiO₂, 36 mol % CaO and 4 mol % P₂O₅.

S70C30: 70 mol % SiO₂, 30 mol % CaO.

Thomas et al., J Long Term Eff Med Implants, 2005; 15(6):585-97 reviews different bioactive glass materials and their uses. The bioactive glass compositions suitable for use in accordance with the present disclosure are not limited to the particular examples provided, but include other bioactive glass materials such as those known in the art. Many bioactive glass compositions are also disclosed in U.S. Pat. Nos. 5,735,942; 6,054,400; 6,171,986 and 6,517,857.

Bioactive and biocompatible glasses have been developed as bone replacement materials. Studies have shown that these glasses will induce or aid osteogenesis in physiologic systems. The bond developed between the bone and the glass has been demonstrated to be extremely strong and stable. However the size of the particles used to form bone is relatively large.

Tooth dentin is very different from bone. The organic component of dentin (approximately 40%) makes most systems that will bond to bone and tooth enamel ineffective. Most current materials used for treatment of desensitization rely on materials that have been optimized for the bonding to bone and tooth enamel by their interaction with the inorganic components. As a result, even the most effective treatments are short lived. Therefore, there is a need in the dental field for a material that would chemically react with the surface of dentin and intimately bond to tooth structure, which would significantly reduce the reopening of dentin tubules due to contact with oral fluids.

The compositions disclosed herein provide for mechanical and chemical obturation of the tubules. Moreover, in certain embodiments, the present compositions and methods actually provide a bioactive layer that will form a new structural layer which results in long-lasting reduction of tooth hypersensitivity. This has been verified by the reformation of a hydroxycarbonate apatite layer on and in dentin surfaces after treatment with compositions disclosed herein, as determined by light and electron microscopy and fluid conductance studies. Particles that are small enough to fit inside or rest on the opening of the tubules provide for actual physical occlusion of the tubules. Thus, embodiments of the disclosed compositions include particles smaller than 90 microns, such particles are more likely to adhere to the tubules or tooth surface because particles less than about 90 microns react quickly enough to chemically bond with dentin surfaces and tubules during the use of the disclosed compositions, including toothpastes, gels or mouthwashes. In certain disclosed compositions, the bioglass particles have an average diameter of less than about 50 microns, such as less than about 20 microns and may fall into a range of from about 0.1 micron to about 10 microns, such as an average diameter of about 2 microns. In some embodiments the compositions include particles of many sizes but have at least about 25% of the particles having a diameter of less than about 5 microns, such as less than about 2 microns.

The current disclosure reveals that nanostructured, bioactive glass particles chemically react under physiological conditions on the exposed tooth surface to form a biologic apatite network that fills and seals open dentin tubules, resulting in a reduction or elimination of permeability. Light and electron microscopy and X-ray microanalysis confirm that nanostructured, bioactive glass particles produced by the sol-gel method (see Hench and West, Chem. Rev. 1990, 90:33-72) from SiO₂, CaO, and P₂O₅, can initiate biomimetic remineralization on exposed dentin surfaces under simulated oral conditions in vitro. Several glass formulations having different levels of solubility and mineralizing potential have been evaluated for this purpose. The nano-sized bioactive glass particles applied directly to tooth surfaces result in a significant reduction in permeability of exposed dentin upon application, as assessed by light and electron microscopy and fluid conductance studies.

This disclosure also addresses the durability of the glass and new mineral formed on the surface when exposed to acidic solutions and to toothbrush abrasion with a dentifrice. It also discloses the suitability of different delivery vehicles in providing desired handling and application characteristics for the agent, while providing initial sealing and subsequent mineralization to provide a permanent seal. The disclosed formulations include a bioactive glass and a carrier comprising wetting agent. The wetting agent improves the wetting or dispersion of the bioactive glass, facilitating delivery of the bioactive glass particles to the dentin tubules. These wetting agents include HEMA (hydroxyethyl methacrylate polymer), glycerol dimethacrylate, polyacrylic acid, peppermint oil, eugenol, fluoride varnish, and mucoadhesive gels. In one embodiment the carrier provides a temporary seal, allowing the bioactive glass particles to penetrate the dentinal tubules without interference from oral fluids. Typically the carrier is washed away and/or is absorbed, leaving bioactive glass and nascent biomimetically formed apatite.

Embodiments of the compositions disclosed herein generally do not require significant time to set while still providing for long lasting occlusion of dentin tubules. Previous compositions of desensitizing agents washed away by mechanical abrasion caused by brushing, exposure to mild acids in food, salivary flow or other liquids which normally come in contact with the teeth. However, the presently disclosed compositions have been able to generally withstand significant agitation, rinsing with water and long term soaking in simulated saliva. Moreover, the disclosed compositions do not require a set time because they begin to chemically react and adhere to dentin surfaces as soon as they come into contact with these surfaces and fluids naturally present in the mouth. Even though the disclosed compositions are effective to reduce tooth sensitivity with a single application, it is likely that multiple applications will be more efficacious.

The disclosed bioactive glass compositions typically are formulated to have a high viscosity to aid adherence of the composition to the teeth or a specific tooth. For example, the compositions typically have a viscosity of at least about 3 centipoise, such as from about 25 to about 250,000 centipoise, or from about 30 to about 25,000 centipoise, such as from about 35 to about 3,500 centipoise.

The oral compositions disclosed herein typically are formulated in the form of toothpastes or gel dentifrices to be brushed on the teeth, or in the form of mouthwashes. However, other delivery systems may also be used. As non-limiting examples, the subject desensitizing agent can be formulated into a tooth powder, dentifrice, mouthwash, lozenge, buccal adhesive patch, oral spray, coatings that adhere to the oral cavity, chewing gum and the like. Such compositions can be formulated as is known to those of skill in the art to achieve the desired remineralization and/or tooth desensitization effect.

In one aspect the delivery system employs a lipid-based carrier, such as a microemulsion or liposome. Dentifrice compositions described herein contain liposomes between about 0.1 and 20% by weight, preferably between about 3 and 10% by weight. Particularly preferred formulations are dentrifice compositions in the form of a paste or gel that comprises 5% by weight of DOPA liposomes. The liposome may also be incorporated into other liposome membrane-compatible materials which can be used to tailor the release characteristics of any materials that the liposomes may carry. The liposomes may also be used to control the rate of in-tubule liposome biodegradation and to control other aspects of liposome stability. Preferably, the liposomes of the present composition are prepared from salts of diolylphosphatidic acid (DOPA, Avanti Polar Lipids, Inc.). To penetrate and be retained in the dentinal tubules, the liposome diameter should not be greater than about 2 microns, typically from about 0.1 to 1.5 microns, and most preferably less than about 0.5 micron. Therefore, in certain embodiments, the present compositions provide a dentifrice for treating hypersensitive teeth including an effective amount of a mineral-inducing liposome wherein the liposome is formed from a salt of DOPA, such as the potassium salt of DOPA, and has a diameter not greater than 0.5 microns.

The formulations disclosed herein may contain additional ingredients typically incorporated into oral health care compositions. Suitable ingredients include, without intended limitation, abrasive polishing materials, sudsing agents, flavoring agents, humectants, binders, water and sweetening agents, in particular, high intensity sweeteners, such as sucralose, aspartame and saccharin. Abrasives which may be used in disclosed compositions include alumina and hydrates thereof, such as alpha alumina trihydrate, magnesium trisilicate, magnesium carbonate, aluminosilicate, such as calcined aluminum silicate and aluminum silicate, calcium carbonate, zirconium silicate, powdered polyethylene, silica xerogels, hydrogels and aerogels and the like. Also suitable as abrasive agents are calcium pyrophosphate, insoluble sodium metaphosphate, calcium carbonate, dicalcium orthophosphate, particulate hydroxyapatite and the like. Depending on the form that the oral composition is to take, the abrasive may be present in an amount up to 70% by weight, preferably 1 to 70% by weight, more preferably from 10 to 70% by weight, particularly when the composition is formulated into a toothpaste.

Humectants contemplated for use in the subject compositions include, without limitation, polyols, such as sorbitol, polyethylene glycols, propylene glycol, hydrogenated partially hydrolyzed polysaccharides and the like. The humectants are generally present in amounts up to 80%, preferably 5 to 70%, by weight for toothpaste formulations. Optional thickeners suitable for use in the disclosed compositions, typically silica or titanium dioxide, may be present at a level from about 0.1 to 20% by weight if present.

Binders suitable for use in the compositions disclosed herein include cellulose derivatives, such as hydroxyethyl cellulose, hydroxypropyl cellulose and hydroxypropyl methylcellulose as well as xanthan gums, Irish moss and gum tragacanth. Binders may be present in the amount from about 0.01 to about 10%. Sweeteners suitable for use, such as saccharin, may be present at levels of about 0.1% to 5%.

Inclusion of fluoride sources in the presently disclosed oral compositions result in apatite fluoride formation during the remineralization initiated by administration of the compositions. Suitable fluoride sources include, without limitation, those commonly used in oral health care compositions, such as sodium fluoride, stannous fluoride, sodium monofluorophosphate, zinc ammonium fluoride, tin ammonium fluoride, calcium fluoride and cobalt ammonium fluoride and the like. Preferred compositions include a fluoride source for the formation of apatite fluoride formation. Fluoride ions are typically provided at a level up to 1500 ppm, preferably 50 to 1500 ppm, although higher levels up to about 3000 ppm may be used as well.

Surfactants, such as a soap, anionic, nonionic, cationic, amphoteric and/or zwitterionic, may be present in amounts up to about 15%, preferably 0.1 to 15%, more preferably 0.25 to 10% by weight. Anionic and/or nonionic surfactants are most preferred, such as sodium lauryl sulfate, sodium lauryl sarcosinate and sodium dodecylbenzene sulfonate. Suitable flavor additives are usually included in low amounts, such as from 0.01 to about 5% by weight, especially from 0.1% to 5%.

In certain embodiments, tooth desensitizing compositions may, and preferably will, include antibacterial agents including, for example, phenolics and salicylamides, and sources of certain metal ions such as zinc, copper, silver and stannous ions, for example, zinc, copper and stannous chloride, and silver nitrate. Such agents, in addition to other functional agents, including therapeutic agents and nutrients, also may be incorporated into the compositions disclosed herein.

Dyes/colorants suitable for oral health care compositions, such as FD & C Blue #1, FD & C Yellow #10, FD & C Red #40, and the like, may be employed in the subject formulations as well. Various other optional ingredients may also be included in the disclosed compositions, including without limitation those such as preservatives, vitamins such as vitamins C and E, and other anti-plaque agents such as stannous salts, copper salts, strontium salts and magnesium salts. Also included may be pH adjusting agents; anti-caries agents such as calcium glycerophosphate, sodium trimetaphosphate; and anti-staining compounds such as silicone polymers, plant extracts and mixtures thereof. Additionally, polymers, particularly anionic polymers, such as polycarboxylates or polysulfonates, or polymers containing both a carboxylate and a sulfonate moiety, phosphonate polymers or polyphosphates may be included. Other optional carrier components fulfill multiple functions, for example, acting both as carriers and flavorants. For example certain carriers include menthol, peppermint oil and/or eugenol. Menthol, peppermint oil and eugenol are examples of carriers as well as being organic flavorants.

The various substances mentioned above are ingredients suitable for oral care compositions, for example, toothpastes, gels, mouthwashes, gums, powders, etc. Except where otherwise noted, references to toothpastes are to be interpreted as applying to gels as well. Mouthwash forms, for example, mouthwashes, oral rinses and similar preparations, may be formulated as well. Such preparations typically comprise a water/alcohol solution, including a flavor component, humectant, sweetener, sudsing agent, and colorant. Mouthwashes can include ethanol at a level of from 0 to 60%, preferably from 5 to 30% by weight.

### EXAMPLE 1

### Bioactive Glass Compositions

Particular examples of bioactive glass compositions that are used include those with 50-90% SiO₂, for example 60-90% or 65-85% SiO₂. In particular examples, the particles were less than less then about 40 μm in size, for example a substantial component of the particles (such as at least 50%) were less than 10 μm.

Three different formulations of bioactive glass with different solubility levels were synthesized:

Bioactive glass 1 (BAG1): 65 mol % SiO₂, 31 mol % CaO, 4 mol % P₂O₅

Bioactive glass 2 (BAG2): 75 mol % SiO₂, 21 mol % CaO, 4 mol % P₂O₅

Bioactive glass 3 (BAG3): 85 mol % SiO₂, 11 mol % CaO, 4 mol % P₂O₅

The expectation was that the 65% glass would be the quickest to react due to its low silica content, and the 85% glass would be the most stable and therefore the least soluble and slowest to react in simulated body fluid (SBF). One method for creating the fine bioactive glass powder for the composition included dry grinding in mortar and pestle and sieving to below 37 μm. This produced particles whose major constituents were below 10 μm, as examined in the scanning electron microscope. The bioactive glass particles could be ground to an even smaller size in some examples by ball milling with ceramic pellets in a slurry of alcohol.

To obtain the highest homogeneity, all starting compounds are high purity metal oxides. Tetraethyl orthosilicate is used as the precursor for silica (SiO₂) in the final glass. Calcium methoxyethoxide (CMOE) is used as the precursor for lime, and triethyl phosphate as the precursor for phosphate. The manufacturer supplies CMOE as a 20% solution in methoxyethanol, and this alcohol serves as a mutual solvent for all of the alkoxide and the water used to initiate hydrolysis and glass formation. By controlling the reaction of the alkoxides with water, a highly homogenous final glass results. The solutions are prepared in a dry nitrogen environment glovebox. After mixing they are cast into polyethylene containers and allowed to hydrolyze undisturbed for time periods up to 3 weeks.

The glass is aged in distilled water, air-dried and stabilized in a dedicated furnace at temperatures up to 600° C., using a temperature ramp and soak sequence over a period of two days. In one protocol, the glass is heated from room temperature to 37° C. and subjected to 100% humidity until a complete gellation reaction has occurred (usually days). After complete gellation, the temperature is raised to 90° C. at a rate of 1.0 degree per minute. This and all subsequent steps are done in air, without controlling humidity. Next, the temperature is held for 120 minutes. Next the temperature is raised to 180° C. at 1.0 degree per minute. After 300 minutes at 180° C., the temperature is increased to 600 degrees at a rate of 2.5 degrees per minute. This temperature is held for 900 minutes. After this time, the sample is removed completely from the furnace cooled by air flow across the dispersed glass pieces. The temperature is dropped as quickly as possible but without immersion into any fluid, only air.

This temperature treatment will completely remove residual alcohols and alkoxide components, yet retains the high surface area of the glasses. The porosity of the glass is controlled with temperatures and times of aging. The glass is therefore prepared at a temperature and for a time that limits particle dimension to less than about 20 μm. This continuum of sizes is desired because it will provide some particles less than 2 μm, which are particularly suited for entering and obstructing tubule openings. In some embodiments the composition contains sufficient particles less than 2 μm to provide an effective desensitizing dosage of the particles. Ground particles are actually composed of nanometer-sized agglomerates, but additional grinding is preferred (using mechanical tituration or ball milling) to obtain particles of the desired size to enter the tubule.

### EXAMPLE 2

### Phosphoric Acid Gel Carrier

Attempts to apply the powder in water directly to dentin surfaces did not create good coverage or retention. It was ultimately shown that incorporating the powder in an acid-based etchant, such as an acid gel (for example phosphoric acid etch gel) produced good coverage. The gel suitably has a viscosity greater than water, but may be sufficiently flowable to be placed on the tooth, and sufficiently viscous (or develops sufficient viscosity) to be retained on the tooth. In a particular example the phosphoric acid gel included 35% phosphoric acid, but 20-45% (such as 30-40%) phosphoric acid may be used in other examples. Light and electron microscopy revealed that the particles partially covered the surface, became embedded on the surface, and entered the dentin tubules to some extent. This penetration of the glass into the tubules was greater than that of the control silica nanoparticles (OX50, Degussa; avg. size=40-50 nm). If allowed to dry, the surface became glazed with the glass. There was some evidence for precipitation of new mineral on the tooth surface, as intended. Both the 65% and 85% BAG have been tested, but a substantial difference was not observed between the results. Hence the 65% formulation has been used throughout the rest of the examples.

### EXAMPLE 3

### Nanostructured Bioactive Glass Reduced Fluid Conductance in Dentinal Tubules

Studies with the most soluble of the glasses show a significant reduction of flow through dentin tubules of more than 50% after application of the glass in etchant. The conductance was measured immediately and at 1, 24, 96 and 168 hours. This reduction was greater than that of the etchant alone or the control (silica nanofillers), and the reduction is maintained for at least up to 7 days.

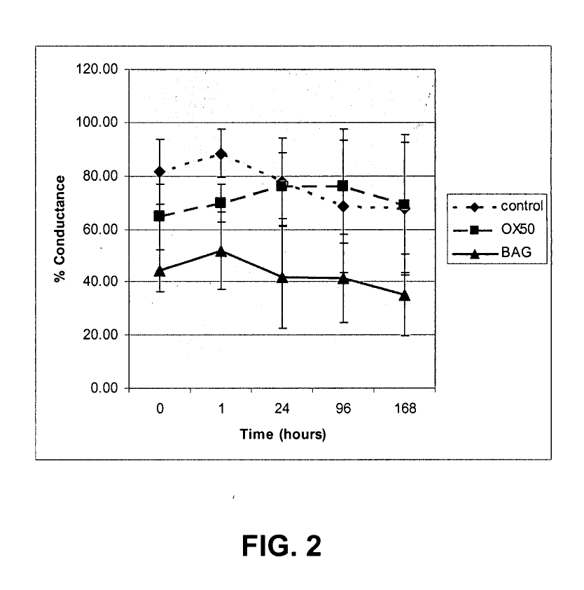

A bioactive glass with nanostructured porosity (BAG-65 mole % SiO₂/11 mole % CaO/4 mole % P₂O₅) that was verified by FTIR to show spontaneous biomimetic apatite production in simulated body fluid (SBF) was produced by the sol-gel method, and ground with mortar and pestle to a fine powder (sieved below 38 μm). Dentin/pulp chamber specimens (n=3-5) were prepared from human teeth, mounted, perfused with SBF, etched with 35% phosphoric acid gel (PA), and then brushed for 60 seconds with either PA (control), 15 wt % OX50 silica nanofiller in PA, or 15 wt % BAG in PA. The hydraulic conductance of the dentin slab was tested after acid etching to open the tubules (original), and then after 0 hour (immediate), 1, 24, 96, and 168 hours after treatment (maintaining perfusion with SBF at 20 cm). Results of fluid conductance (% of original) were analyzed with 2-way ANOVA/Tukey's (p<0.05). Results: There was no interaction, and the effect of time was not significant. BAG showed a significant reduction in fluid conductance in the dentin at each time period versus the control, and at 24 and 168 hours versus OX50. BAG was effective at producing an immediate reduction in fluid conductance, and maintaining it for at least 7 days. SEM analysis showed evidence of finely ground BAG particles covering the dentin surface and occluding tubules. Thus the presently disclosed, finely ground, nanostructured bioactive glass has been demonstrated to be an effective biomimetic dentin desensitizer. The data is shown in FIG. 2.

### EXAMPLE 4

### Carriers for Applying Bioactive Glass Desensitizers

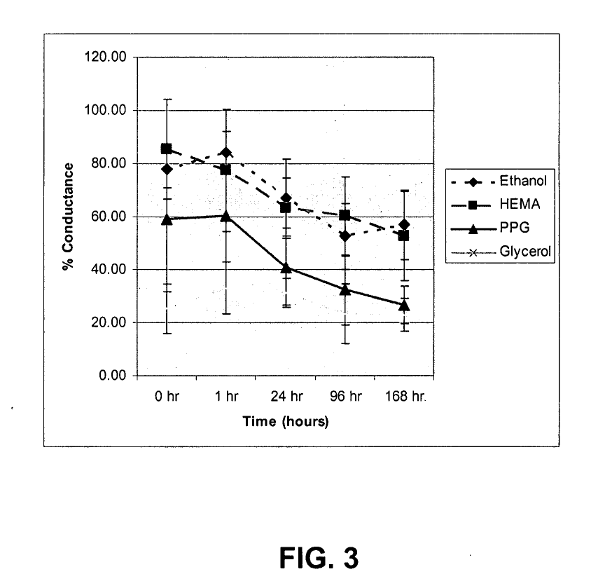

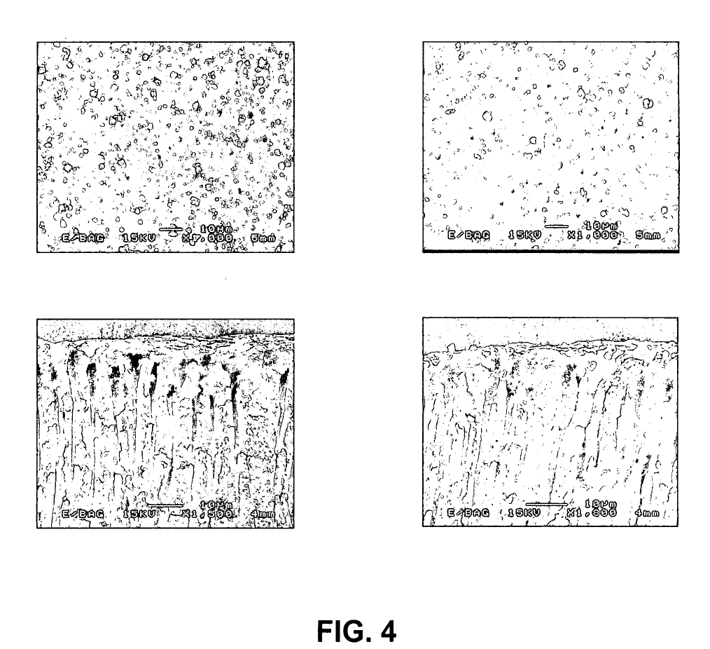

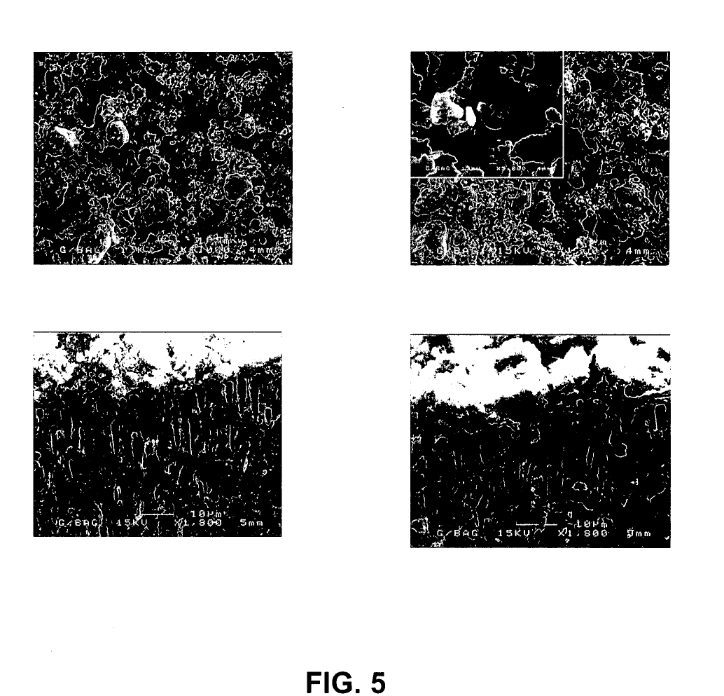

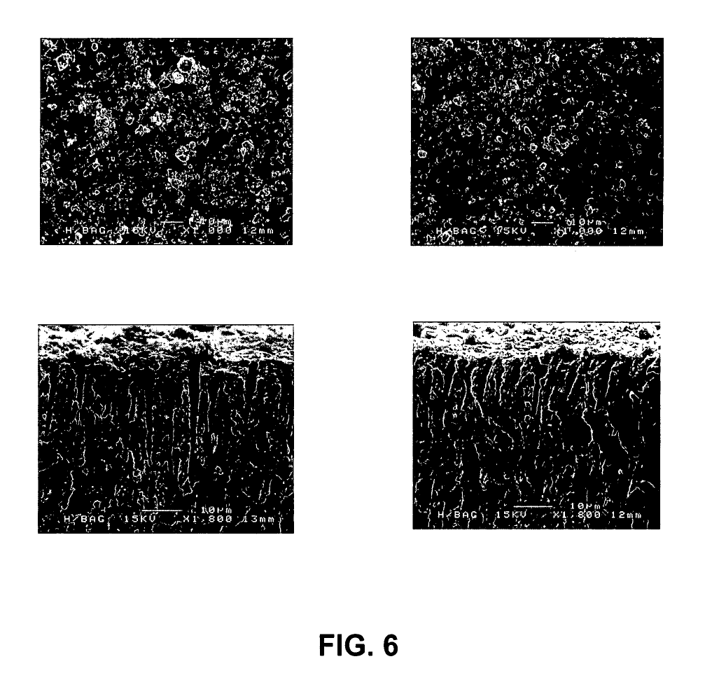

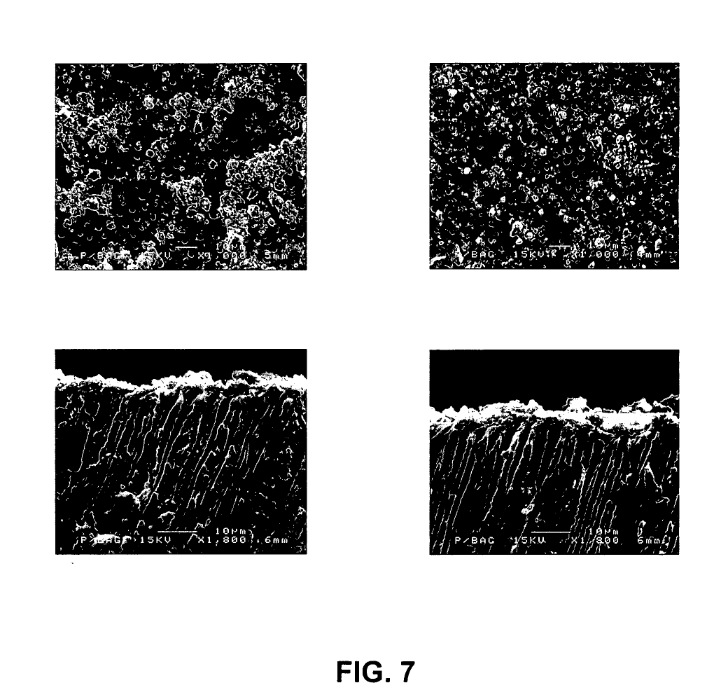

This example provides data for the use of application carriers for the BAG in durability experiments. The carriers used in the examples were ethanol, propylene glycol, glycerol, and HEMA. All of these agents were expected to wet the tooth surface well, due to their hydrophilicity, and could be filled with BAG to the 15 wt % level and still be easily dispensed and spread onto the dentin surface. The dentin surface was first treated with 35% phosphoric acid to open the tubules. The hydraulic conductance was measured at 20 cm pressure (with simulated body fluid as the perfusion medium), and then the desensitizer in the various carriers were applied to the dentin surface. The hydraulic conductance was remeasured with the tooth kept under pressure. The results in FIG. 3 showed that the glycerol and the propylene glycol were the most effective carriers in terms of reducing fluid flow, and the glycerol was overall the best because it maintained the same level of flow reduction from the moment it was applied for up to 7 days. SEM micrographs of representative surfaces showed evidence for tubule occlusion and general coating of the surface with the BAG in the four carriers, as illustrated in FIGS. 4-7.

### EXAMPLE 5

### BAG Induced Reduction in Conductance Continued after Brushing

Glycerol with 15 wt % BAG was used for the durability demonstrations, since glycerol was shown to be the best carrier. The dentin surfaces were exposed to the desensitizer and then subjected to toothbrushing (5 minutes at 1 Hz) with a common dentifrice in a custom toothbrushing machine (specimens rotating on a wheel in contact with the brush and being dipped into a dentifrice slurry). After brushing, the specimens were evaluated for conductance, and then exposed to the test solutions (Coca Cola Classic, grape fruit juice, and coffee) in sequences by dipping them into the solution at a frequency of 1 Hz for 5 minutes. The control group did not have BAG applied to its surface, but was exposed to the brushing, and the solutions. The results showed that the reduction in conductance due to the BAG continued even after the brushing and exposure to the three solutions.

The dentin desensitizing agent containing bioactive glass was shown to be effective at reducing conductance of fluids through patent dentin tubules in vitro, and was generally stable after being exposed to routine toothbrush abrasion and various drinks (cola, juice and coffee). The agent was most effectively applied to dentin in a glycerol carrier, which showed effective coverage of the surface and blockage of dentinal tubules.

In view of the many possible embodiments to which the principles of our invention may be applied, it should be recognized that the illustrated embodiments are only examples of the invention and should not be taken as a limitation on the scope of the invention. Rather, the scope of the invention is defined by the following claims. We therefore claim as our invention all that comes within the scope and spirit of these claims.

We claim:

1. A dental composition for treating dentinal hypersensitivity, comprising a particulate bioactive glass; and a carrier, wherein the bioactive glass comprises
from about 50 to about 96 mole percent SiO₂;
from about 2 to about 50 mole percent CaO;
from about 2 to about 16 mole percent P₂O₅; and
wherein the carrier comprises a wetting agent capable of delivering the bioactive glass to a dentinal tubule, the carrier having a viscosity of at least about 3 centipoise.

2. The composition of claim 1, wherein the wetting agent is a hydrophilic wetting agent.

3. The composition of claim 1, wherein the wetting agent comprises a liposome.

4. The composition of claim 3, wherein the liposome has a diameter of from about 0.1 to about 0.5 microns.

5. The composition of claim 1, wherein the carrier has a viscosity of from about 25 to about 250,000 centipoise.

6. The composition of claim 1, wherein the carrier has a viscosity of from about 30 to about 25,000 centipoise.

7. The composition of claim 2, wherein the hydrophilic wetting agent comprises at least one of hydroxyethyl methacrylate polymer (HEMA), polyacrylic acid, polyacrylic acid/itaconic acid copolymer, phosphoric acid, polyacrylic acid/maleic acid copolymer, glycerol, propylene glycol, ethanol and polyglutamic acid.

8. The composition of claim 1, wherein the carrier comprises an organic flavorant.

9. The composition of claim 8, wherein the organic flavorant comprises menthol, peppermint oil, eugenol or a combination thereof.

10. The composition of claim 1, wherein the carrier comprises eugenol.

11. The composition of claim 1, wherein the bioactive glass further comprises from about 0.1 to about 10 mole percent of a borate.

12. The composition of claim 8, wherein the borate is B₂O₃

13. The composition of claim 1, wherein the bioactive glass further comprises from about 0.1 to about 25 mole percent of a fluoride.

14. The composition of claim 13, wherein the fluoride is CaF₂

15. The composition of claim 1, wherein the bioactive glass comprises particles having an average diameter of less than about 50 microns.

16. The composition of claim 1, wherein the bioactive glass comprises particles having an average diameter of less than about 20 microns.

17. The composition of claim 1, wherein the bioactive glass comprises particles having an average diameter of from about 0.1 micron to about 10 microns.

18. The composition of claim 15, wherein at least about 25% of the particles have a diameter of less than about 5 microns.

19. The composition of claim 1, wherein the bioactive glass comprises particles having an average diameter less than about 2 microns.

20. A bioactive glass composition for oral administration, comprising the composition of claim 1 and a toothpaste, fluoride varnish, glycerin gel or mouthwash.

21. A method for at least partially occluding dentin tubules comprising contacting said tubules with the bioactive glass composition of claim 20.

22. A method for treating tooth hypersensitivity in a subject in need thereof, comprising administering to the subject the composition of claim 1, wherein at least a portion of the bioactive glass is covalently incorporated into the subject's dentin.
